(19) United States
(12) Patent Application Publication
(10) Pub. No.: US 2012/0035645 A1
(43) Pub. Date: Feb. 9, 2012

(54) DYNAMIC AND STATIC BLOOD FILTERS

(75) Inventor: Yossi GROSS, Moshav Mazor (IL)

(73) Assignee: RAINBOW MEDICAL LTD., Herzliya (IL)

(21) Appl. No.: 12/851,263

(22) Filed: Aug. 5, 2010

(51) Int. Cl. A61F 2/01

(52) U.S. Cl. 606/200

(57) ABSTRACT

Apparatus and methods are described for preventing stroke. The apparatus includes a housing, and a blood rotation device coupled to the housing. The blood rotation device facilitates the preventing of the stroke by rotating blood flowing through an artery of a subject, without the device storing energy during systole of the subject that is subsequently released by the device during diastole of the subject. Other embodiments are also described.

### FIELD OF EMBODIMENTS OF THE INVENTION

The present invention generally relates to medical apparatus. Specifically, the present invention relates to medical apparatus for preventing the occurrence of stroke.

### BACKGROUND

A stroke occurs when a portion of the brain is deprived of blood flow. A common cause of stroke is the migration of material in systemic blood vessels to the brain. An embolus for example, often arises from the heart and lodges in a cerebral blood vessel. Embolic material typically includes blood clots and atherosclerotic debris, in addition to foreign material introduced into the circulation.

U.S. Pat. No. 6,953,438 to Milo describes a number of ultrasonic devices for preventing microbubbles and/or microparticles from reaching the brain during a PCI, or cardiovascular surgery. Devices are designed for implantation in the chest cavity and operate in combination with needle vents or other vent systems for removing diverted microbubbles. Systems are designed for noninvasive employment. Devices are described as being particularly designed to prevent microbubbles from reaching the great origins of the carotid arteries and/or for diverting bubbles that might reach the vicinity and otherwise pass through. Devices are described as separating microbubbles from a flowing bloodstream and producing a cleansed stream.

U.S. Pat. No. 7,544,160 to Gross describes apparatus including an element shaped so as to define at least one surface. The element is adapted to be implanted in an ascending aorta of a subject in a vicinity of an aortic valve, and, during systole, to store, as potential energy, work applied to the surface by blood of the subject passing through the element. The element allows at least 75% of the blood passing into the element during systole of each cardiac cycle to exit the element during systole of the respective cardiac cycle. The element applies the stored energy to push blood towards at least one coronary artery of the subject during diastole. For some applications, the surface includes a first surface, and the element is shaped so as to define a second surface, which is configured to apply the stored energy. Other embodiments are also described.

The following references may be of interest:
U.S. Pat. No. 6,976,996 to Aboul-Hosn et al.
US Patent Application Publication 2007/0093744 to Elmaleh et al.
PCT Publication WO 06/120464 to Pearce et al.

### SUMMARY OF EMBODIMENTS

For some applications of the present invention, a blood rotation device is disposed within an artery of a subject to prevent the occurrence of a stroke in the subject. Typically, the blood rotation device is placed in the ascending aorta or the carotid artery of the subject.

For some applications, the blood rotation device rotates the blood flowing through it, and diverts emboli within the blood away from the brain of the subject. For some applications of the present invention, the blood rotation device is disposed in the aorta, and diverts emboli within the blood away from the carotid arteries of the subject. Alternatively or additionally, the blood rotation device is disposed within a carotid artery, and diverts emboli within the blood into the subclavian artery of the subject. In either case, the emboli are diverted toward an artery which does not supply the brain, such that stroke is prevented and overall damage due to the emboli is reduced.

Typically, the blood rotation device is implanted in the subject and configured for chronic use. Alternatively, the blood rotation device is designated for acute use, being placed in the carotid artery or aorta during a medical procedure in order to divert emboli released during that procedure, and subsequently being withdrawn from the patient's body at the end of the procedure.

The orientation of the blood rotation device within the artery determines the direction of movement of the emboli due to rotation by the blood rotation device. For some applications, the rotation device is used within the aorta, and the longitudinal axis of the blood rotation device is oriented generally perpendicularly to the longitudinal axis of the aorta, such that the device diverts emboli within the blood away from the carotid artery of the subject. Alternatively or additionally, the rotation device is used within a carotid artery, and the longitudinal axis of the blood rotation device is oriented generally parallel to the longitudinal axis of the carotid artery, such that the device diverts emboli into the subclavian artery of the subject.

A control unit, typically positioned outside the subject's body, drives the blood rotation device to rotate the subject's blood. In one application, a catheter is used to connect the external control unit to the implanted blood rotation device. Alternatively, the blood rotation device operates passively, without an external power source, whereby the rotation is powered by movement of the subject's blood.

There is therefore provided, in accordance with some applications of the present invention, apparatus for preventing stroke, including:

a housing; and

a blood rotation device, coupled to the housing and configured to facilitate the preventing of the stroke by rotating blood flowing through an artery of a subject, without the device storing energy during systole of the subject that is subsequently released by the device during diastole of the subject.

For some applications, the blood rotation device is configured to facilitate the preventing of the stroke by receiving energy from the blood flowing through the artery, and using the energy to rotate the blood.

For some applications, the rotation device is configured to be chronically implanted in the artery.

For some applications, the rotation device is configured for acute use.

For some applications, the rotation device is configured to rotate the blood substantially unidirectionally for a duration of a cardiac cycle of the subject.

For some applications, the rotation device is configured to rotate the blood unidirectionally for a duration of a cardiac cycle of the subject.

For some applications, the rotation device is configured to be disposed within the artery of the subject.

For some applications, the rotation device is configured to be disposed within an aorta of the subject.

For some applications, the rotation device is configured to divert emboli in the blood away from a carotid artery of the subject.

For some applications, the rotation device is configured to rotate the blood with respect to a longitudinal axis of the rotation device, and the rotation device is configured to be disposed within the aorta such that the longitudinal axis of the rotation device is not parallel with a local longitudinal axis of the aorta.

For some applications, the rotation device is configured to be disposed within the aorta such that the longitudinal axis of the rotation device diverges by greater than 75 degrees from the local longitudinal axis of the aorta.

For some applications, the rotation device is configured to be disposed within a carotid artery of the subject.

For some applications, the rotation device is configured to divert emboli in the blood into a subclavian artery of the subject.

For some applications, the rotation device is configured to rotate the blood with respect to a longitudinal axis of the rotation device, and the rotation device is configured to be disposed within the carotid artery such that the longitudinal axis of the rotation device is substantially parallel with a local longitudinal axis of the carotid artery.

For some applications, the rotation device is configured to be disposed within the carotid artery such that the longitudinal axis of the rotation device diverges by less than 15 degrees from the local longitudinal axis of the carotid artery.

For some applications, the apparatus further includes a control unit, configured to facilitate the preventing of the stroke by driving the rotation device to rotate the blood.

For some applications, the control unit is configured to drive the rotation device from a position outside a body of the subject.

For some applications, the apparatus further includes a catheter configured for insertion into the artery of the subject, and the control unit is configured to be coupled to the implanted rotation device via the catheter.

There is further provided, in accordance with some applications of the present invention, apparatus, including:

a housing; and

a blood rotation device, coupled to the housing, configured to rotate blood flowing through an artery of a subject and divert emboli away from a carotid artery of the subject, by rotating the blood, without storing energy in the device during systole of the subject that is subsequently released during diastole of the subject.

For some applications, the blood rotation device is configured to divert the emboli by: receiving energy from the blood flowing through the artery, and using the energy to rotate the blood.

For some applications, the apparatus further includes a control unit, configured to drive the blood rotation device to rotate blood flowing through an artery of a subject.

For some applications, the blood rotating device is configured to be chronically implanted in the artery.

For some applications, the rotation device is configured to rotate the blood substantially unidirectionally for a duration of a cardiac cycle of the subject.

For some applications, the rotation device is configured to rotate the blood unidirectionally for a duration of a cardiac cycle of the subject.

There is additionally provided, in accordance with some applications of the present invention, a method for preventing stroke, including:

identifying a subject at risk of stroke; and

in response to the identifying, rotating blood flowing through an artery of the subject, without storing energy during systole of the subject and subsequently releasing the stored energy during diastole of the subject.

There is further provided, in accordance with some applications of the present invention, apparatus including:

a housing; and

a blood rotation device, coupled to the housing, configured to rotate blood flowing through a first artery of a subject and, at least during systole of the subject, divert the blood into a second artery of the subject by rotating the blood.

For some applications, the device is configured to rotate the blood without storing energy during systole of the subject and subsequently releasing the stored energy during diastole of the subject.

For some applications, the blood rotation device is configured to: receive energy from the blood flowing through the first artery, and use the energy to rotate the blood.

For some applications, the second artery includes a renal artery of the subject, and the blood rotation device is configured to divert blood into the renal artery.

For some applications, the apparatus further includes a control unit configured to drive the blood rotation device to rotate the blood.

For some applications, the blood rotation device is configured to be chronically implanted in the first artery of the subject.

For some applications, the first artery includes an aorta of the subject, and the blood rotation device is configured to be chronically implanted in the aorta.

There is further provided, in accordance with some applications of the present invention, a method including:

implanting a blood rotation device into a first artery of a subject; and

diverting blood into a second artery of the subject during systole of the subject, by rotating the blood.

There is further provided, in accordance with some applications of the present invention, apparatus including:

a housing; and

a blood diversion flap coupled to the housing, configured to divert emboli in blood flowing through a first artery of a subject away from a second artery.

For some applications, the blood diversion flap is configured to passively divert the emboli in the blood.

For some applications, the housing is configured to be disposed within a body of the subject only for the duration of a medical procedure.

For some applications, the housing includes a stent.

For some applications, the housing is configured to be chronically implanted in the first artery of the subject.

For some applications, the housing is configured to be chronically implanted in an aorta of the subject.

There is additionally provided, in accordance with some applications of the present invention, a method, including:

implanting a blood diversion device in a first artery of a subject; and

diverting emboli in blood flowing through the first artery of a subject away from a second artery, using the blood diversion device.

The present invention will be more fully understood from the following detailed description of embodiments thereof, taken together with the drawings, in which:

### BRIEF DESCRIPTION OF THE DRAWINGS

FIG. 1 is a schematic illustration of a blood rotation device disposed within the carotid artery of a subject, in accordance with some applications of the present invention;

FIG. 2 is a schematic illustration of a blood rotation device, disposed within an artery of a subject, in accordance with some applications of the present invention;

FIG. 3 is a schematic illustration of a blood rotation device, disposed within the aorta of a subject, adjacent to a renal artery of the subject, in accordance with some applications of the present invention; and

FIG. 4 is a schematic illustration of a static blood diversion device disposed within the aorta, in accordance with another application of the present invention.

### DETAILED DESCRIPTION OF EMBODIMENTS

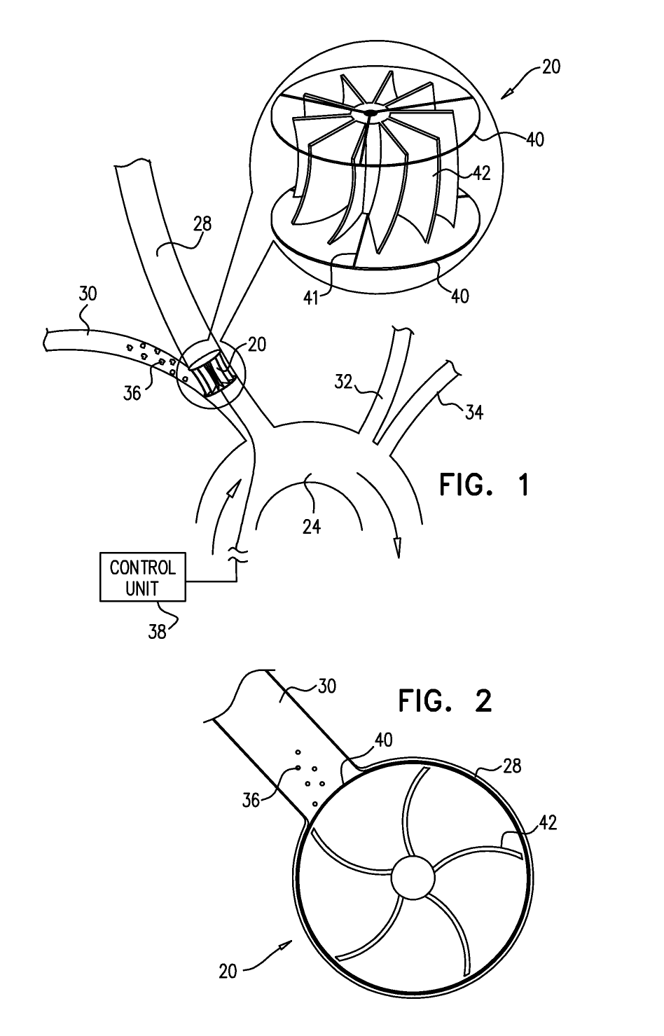

Reference is now made to FIGS. 1 and 2, which are schematic illustrations of blood rotation device 20 disposed within right carotid artery 28, in accordance with some applications of the present invention. Blood rotation device 20 rotates blood flowing through carotid artery 28 and diverts emboli 36 within the blood into subclavian artery 30 of the subject, so as to reduce the overall damage to the subject due to the emboli.

Blood rotation device 20 is oriented within carotid artery 28 so as to enable the diversion of emboli 36 in the blood into subclavian artery 30. Blood rotation device 20 is typically positioned substantially parallel to the longitudinal axis of carotid artery 28, as shown. (Typically, the longitudinal axis of the blood rotation device is positioned at an angle of less than 15 degrees from the local longitudinal axis of carotid artery 28.) Blood rotation device 20 typically rotates the blood with respect to the longitudinal axis of the blood rotation device.

For some applications, blood rotation device 20 is chronically implanted in carotid artery 28, and configured for chronic use in carotid artery 28. Blood rotation device 20 is kept in position within carotid artery 28 by pressing supporting rings 40 surrounding blood rotating device 20 into the wall of carotid artery 28. Typically, blood rotation device 20 is coupled to rings 40 via one or more arms 41.

For some applications, instead of being chronically implanted, device 20 is placed in carotid artery 28 during a medical procedure, and withdrawn at the end of the procedure. For some applications, device 20 is temporarily placed inside the subject's body, for example, for a period of less than two weeks.

Typically, blood rotation device 20 is powered passively, by blood flow through the blood rotation device causing blades 42 of the device to rotate. The rotation of the blades causes radial motion of the blood, which diverts the blood into the subclavian artery (or another artery, depending on implantation site). Further typically, the device does not accumulate energy (e.g., during systole) that is subsequently used to power rotation of the blades (e.g., during diastole). Rather, at any time, the blades are driven to rotate by the blood that is flowing through the device at that time. For example, during diastole, rotation of the blades is powered substantially by the blood that is flowing through the device during diastole.

Typically, blood flow through the artery in which the device is placed is unidirectional in a downstream direction, resulting in the rotation of the blades being unidirectional. For some applications, the device is placed in an artery (such as the aorta) in which there is some backflow of blood in the upstream direction. Even in such arteries, the rotation of the blades of the blood rotation device is typically substantially unidirectional.

For some applications, in which blood rotation device 20 is chronically implanted, a control unit 38 is placed within the subject's body, e.g., using techniques known in the art for implanting the pulse generator of a cardiac pacemaker. The control unit is coupled by leads or wirelessly to blood rotation device 20, in order to drive device 20. For some applications, in which device 20 is placed for an acute treatment and is subsequently withdrawn, control unit 38 is positioned outside of the subject's body, and the control unit drives blood rotation device 20 via leads disposed within a catheter. However, as stated hereinabove, device 20 is typically powered passively, due to blood flow through the device. For such applications, control unit 38 is typically not used to power the device.

For some applications, the control unit detects the subject's cardiac cycle and diverts emboli, by driving blood-rotation-device 20 to rotate the subject's blood during a given phase of the cardiac cycle, e.g., during systole.

For some applications, blood rotation device 20 is disposed within the aortic arch 24 of a subject. Blood rotation device 20 diverts emboli 36 in the blood away from the brain of the subject, toward the descending aorta 26 of the subject, in order to prevent the occurrence of stroke.

Typically, blood rotation device 20 is oriented within aortic arch 24 such that the device diverts emboli 36 away from the right and left carotid arteries 28 and 32, and/or a subclavian artery 34 of a subject. Thus, the longitudinal axis of blood rotation device 60 is typically oriented within aortic arch 24 at an angle greater than 75 degrees from the local longitudinal axis of aortic arch 24 of a subject, for example, 90 degrees from the local longitudinal axis of aortic arch 24.

For some applications, blood rotation device 20 is implanted in the subject, and configured for chronic use in aortic arch 24 of the subject. Blood rotation device 20 is kept in position within the aorta of the subject by pressing supporting rings 40 of the rotation device into the wall of aortic arch 24. Alternatively, device 20 is placed in the aortic arch 24 during a medical procedure, and withdrawn at the end of the procedure.

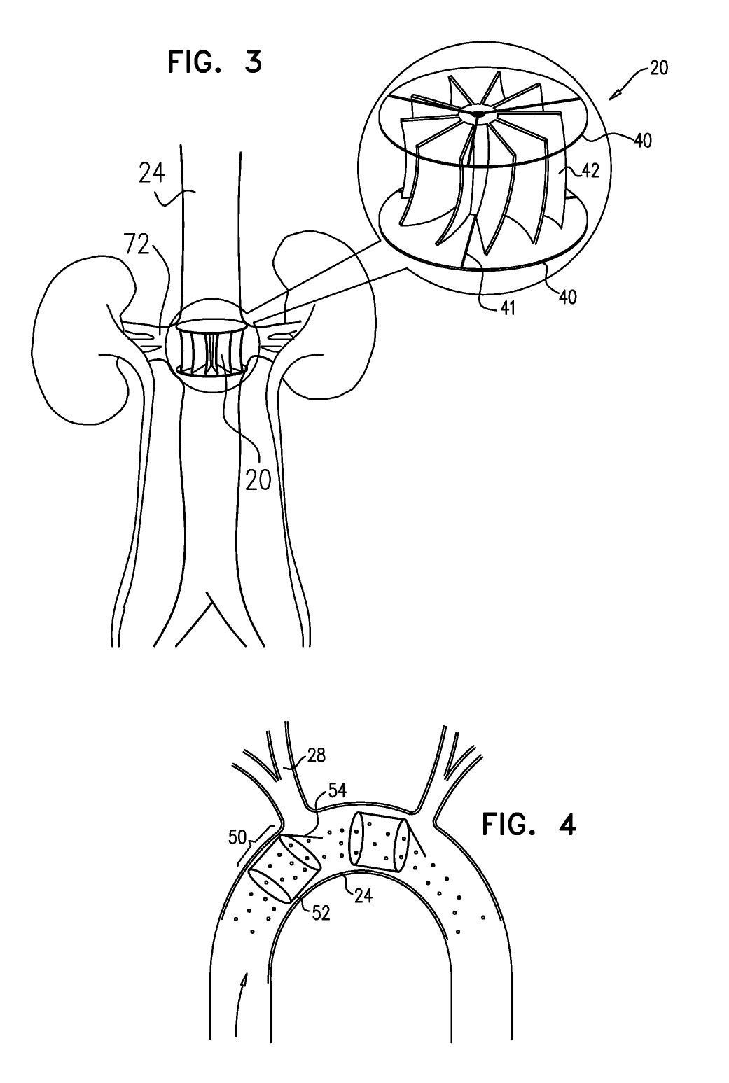

Reference is now made to FIG. 3, which is a schematic illustration of a blood rotation device 20 disposed within aorta 24 of the subject, adjacent to a renal artery 72 of the subject, in accordance with some applications of the present invention. For some applications, blood rotation device 20 is positioned adjacent to renal artery 72 of the subject in order to enhance kidney perfusion. Typically, a portion of the energy of the blood flow in aorta 24 is used to rotate blades 42 of blood rotation device 20, such that rotation of the blood causes radial motion of a portion of the blood, diverting the blood into renal artery 72. The increase in renal blood flow is accompanied by an identical decrease in aortic blood flow distal to device 20, but the relative increase in renal blood flow is higher than the relative decrease in aortic blood flow.

Reference is now made to FIG. 4, which is a static blood diversion device 50, disposed within aorta 24 of a subject, in accordance with an alternative application of the present invention. Blood diversion device 50 typically comprises a housing 52 (e.g., a stent) and a blood diversion flap 54 coupled to housing 52. Flap 54 is positioned at an angle with respect to the local longitudinal axis of aorta 24 (or, in other applications, another artery in which device 50 is placed). Typically, flap 54 diverts emboli away from an adjacent artery (carotid artery 28 in the figure), while allowing blood to flow with fewer emboli to the adjacent artery. As appropriate for any given application, blood diversion device 50 may be chronically implanted or placed within the body temporarily, e.g., for the duration of a medical procedure. Although, device 50 is shown inside the aorta, for some applications the device is placed in a different artery of the subject, mutatis mutandis.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for preventing stroke, comprising:
a housing; and
a blood rotation device, coupled to the housing and configured to facilitate the preventing of the stroke by rotating blood flowing through an artery of a subject, without the device storing energy during systole of the subject that is subsequently released by the device during diastole of the subject.

2. The apparatus according to claim 1, wherein the blood rotation device is configured to facilitate the preventing of the stroke by receiving energy from the blood flowing through the artery, and using the energy to rotate the blood.

3. The apparatus according to claim 1, wherein the rotation device is configured to be chronically implanted in the artery.

4. The apparatus according to claim 1, wherein the rotation device is configured for acute use.

5. The apparatus according to claim 1, wherein the rotation device is configured to rotate the blood substantially unidirectionally for a duration of a cardiac cycle of the subject.

6. The apparatus according to claim 1, wherein the rotation device is configured to rotate the blood unidirectionally for a duration of a cardiac cycle of the subject.

7. (canceled)

8. The apparatus according to claim 1, wherein the rotation device is configured to be disposed within an aorta of the subject.

9. The apparatus according to claim 8, wherein the rotation device is configured to divert emboli in the blood away from a carotid artery of the subject.

10. The apparatus according to claim 8, wherein the rotation device is configured to rotate the blood with respect to a longitudinal axis of the rotation device, and wherein the rotation device is configured to be disposed within the aorta such that the longitudinal axis of the rotation device is not parallel with a local longitudinal axis of the aorta.

11. (canceled)

12. The apparatus according to claim 1, wherein the rotation device is configured to be disposed within a carotid artery of the subject.

13. The apparatus according to claim 12, wherein the rotation device is configured to divert emboli in the blood into a subclavian artery of the subject.

14. The apparatus according to claim 12, wherein the rotation device is configured to rotate the blood with respect to a longitudinal axis of the rotation device, and wherein the rotation device is configured to be disposed within the carotid artery such that the longitudinal axis of the rotation device is substantially parallel with a local longitudinal axis of the carotid artery.

15-24. (canceled)

25. A method for preventing stroke, comprising:
identifying a subject at risk of stroke; and
in response to the identifying, rotating blood flowing through an artery of the subject, without storing energy during systole of the subject and subsequently releasing the stored energy during diastole of the subject.

26. (canceled)

27. The method according to claim 25, wherein rotating the blood comprises receiving energy from the blood flowing through the artery, and rotating the blood using the received energy.

28. The method according to claim 25, wherein rotating the blood comprises rotating the blood substantially unidirectionally for the duration of a cardiac cycle of the subject.

29. The method according to claim 25, wherein rotating the blood comprises rotating the blood unidirectionally for the duration of a cardiac cycle of the subject.

30. The method according to claim 25, wherein rotating blood comprises rotating blood flowing through an aorta of the subject.

31. The method according to claim 30, wherein rotating blood comprises diverting emboli within the blood away from a carotid artery of the subject.

32. The method according to claim 25, wherein rotating blood comprises rotating blood flowing through a carotid artery of the subject.

33. The method according to claim 32, wherein rotating blood comprises diverting emboli within the blood toward a subclavian artery of the subject.

34-40. (canceled)

41. A method comprising:
implanting a blood rotation device into a first artery of a subject; and
diverting blood into a second artery of the subject during systole of the subject, by rotating the blood.

42-59. (canceled)
